(19) United States
(12) Patent Application Publication
(10) Pub. No.: US 2021/0204552 A1
(43) Pub. Date: Jul. 8, 2021

(54) ANTIMICROBIAL COMPOSITION

(71) Applicant: Nordic Biotech Group Oy, Espoo (FI)

(72) Inventor: Kari Holopainen, Lahti (FI)

(73) Assignee: Nordic Biotech Group Oy, Espoo (FI)

(21) Appl. No.: 17/055,926

(22) PCT Filed: May 10, 2019
(86) PCT No.: PCT/FI2019/050374

§ 371 Date: Nov. 16, 2020

(30) Foreign Application Priority Data
May 16, 2018 (FI) 20185450

(51) Int. Cl. A01N 65/06; A01N 25/02

(52) U.S. Cl. CPC A01N 65/06; A01N 25/02

(57) ABSTRACT

An antimicrobial composition of coniferous resin acids and solvent, such as alcohol, water, and an auxiliary solvent selected from E and P series glycol ethers is disclosed. The method for manufacturing of the antimicrobial composition is also disclosed, where in the first step coniferous resin acids, alcohol, auxiliary solvent and water are provided; in the second step alcohol is mixed with water; in the third step coniferous resin acids, auxiliary solvent, as well as optionally a wetting agent, water and/or a pH regulator are added into the alcohol-water solution from second step, and mixed until clear; in the fourth step the antimicrobial composition is optionally packaged and/or diluted to obtain an alcoholic antimicrobial composition with coniferous resin acid concentrations more than 0.01 weight-%. The disclosure relates further to the use of the antimicrobial composition as disinfectant for inanimate and/or living surfaces.

### FIELD OF THE DISCLOSURE

The disclosure relates to an antimicrobial composition, and particularly to an antimicrobial composition comprising coniferous resin acids. The present disclosure further relates to a process for producing antimicrobial composition, and to the use of said antimicrobial composition.

### BACKGROUND OF THE DISCLOSURE

Antimicrobial compositions typically comprise different components depending on the intended purpose of use. For example, disinfectants for use on inanimate surfaces may comprise chlorine compounds, such as sodium hypochlorite and chlorhexidine, metal compounds and/or different types of aldehydes, alcohols, phenol compounds, quaternary ammonium compounds, halogen compounds, peroxides, hydroperoxides etc. Furthermore, many disinfectant compositions comprise compounds that may cause allergic reactions even if not applied to the skin because of the potential for skin contact with such inanimate surfaces. Antimicrobial metal compounds, such as silver and copper, may cause contact eczema, and chlorine compounds although commonly used in disinfectants are potentially allergenic substances that have irritating properties, such as skin, eye and respiratory irritation properties. Said compounds may also cause danger to the environment due to their toxicity.

Antimicrobial compositions, and especially disinfectants are compositions that typically provide only short-term protection against bacteria and/or viruses, and the most commonly used antimicrobial compounds are typically highly volatile compounds, such as alcohols that evaporate rapidly after application on the surface to be treated. Although some disinfectant formulas contain compounds, such as silver, that stay on the treated surface after other compounds have drained away, these formulas may cause allergic reactions and therefore are not suitable for use as disinfectants for living surfaces. Furthermore, silver is also harmful to the environment, and interferes with the operation of the activated sludge.

WO2006098651 discloses compositions for disinfecting inanimate surfaces, wherein the disinfectant is selected from the group consisting of aldehydes, alcohols, phenol compounds, quaternary ammonium compounds, chlorhexidine, halogen compounds, peroxides and hydroperoxides.

UA89422 (U) discloses a process for the preparation of disinfectant of prolonged action for sterilization of surfaces based on interaction of finely dispersed silver metal and aqueous-alcoholic solution. The aqueous-alcoholic solution contains 30% of alcohol, 64% of water and 6% of finely dispersed silver metal.

US2013071488 discloses a disinfectant composition for hard articles, which includes a first agent containing a powder mixture (A) and a second agent containing an aqueous hydrogen peroxide solution (B-1), the powder mixture (A) containing an alkali metal salt (A-1) exhibiting basicity when the salt is in the form of an aqueous solution, a water-soluble copper salt (A-2), a compound (A-3) represented by the formula (1), and a nonionic surfactant (A-4) represented by the following formula (2), and in which the molar ratio of the water-soluble copper salt (A-2) and the compound (A-3) represented by the mixing amount of (A-3)/mixing amount of (A-2) is 3.0 to 20; and a single-agent type disinfectant composition for hard articles, which includes the components (A-1) to (A-4), and an inorganic peroxide (B-2) that releases hydrogen peroxide in water, and in which the molar ratio of the water-soluble copper salt (A-2) and the compound (A-3) represented by the mixing amount of (A-3)/mixing amount of (A-2) is 3.0 to 20.

Resin, such as spruce resin, has been used for years in antimicrobial compositions, such as ointments and salves, for example in folk medicine. Said compositions however, may contain many impurities, and therefore the resulting mixtures are non-homogenous. Furthermore, content of the coniferous resin acids in said compositions is difficult to standardize as the content of the coniferous resin acids in the spruce resin varies a lot.

Another challenge in the manufacturing of antimicrobial compositions containing coniferous resin acids is that coniferous resin acids are poorly water-soluble, and therefore coniferous resin acids are typically dissolved in solvents, such as methanol, acetone and diethyl ether, or they may be melted and processed at high temperatures.

Document WO2011042613 discloses an antimicrobial composition comprising coniferous resin acids and/or their derivatives dissolved in a suitable solvent, such as methanol, ethanol, isopropanol, acetone, ether, chloroform or formaldehyde.

At present there is a growing need to develop new antimicrobial compositions for use in killing harmful bacteria, because the amount and prevalence of antibiotic resistant microbes is constantly increasing. Furthermore, antimicrobial compositions are needed, which do not cause harm to the environment or animals.

### BRIEF DESCRIPTION OF THE DISCLOSURE

An object of the present disclosure is to provide an antimicrobial composition comprising coniferous resin acids to overcome the above problems. Another object of the present disclosure is to further provide a process for producing antimicrobial composition and use of said antimicrobial composition as disinfectant for inanimate and living surfaces.

The objects of the disclosure are achieved by a composition, method of manufacturing said composition and use of said antimicrobial composition which are characterized by what is stated in the independent claims. The preferred embodiments of the disclosure are disclosed in the dependent claims.

A problem with the existing disinfectant compositions is that said disinfectants tend to contain allergy causing ingredients and/or ingredients as well as nonbiodegradable ingredients harmful to the environment, such as silver and quaternary ammonium compounds. Coniferous resin acids are also poorly water soluble, and therefore they tend to separate from the compositions resulting in an uneven distribution on the treated surfaces. Furthermore, for example alcoholic disinfectants are easily volatile and thus their disinfecting effect only lasts for a short period of time.

The disclosure is based on the surprising finding that an improved antimicrobial composition can be obtained by an antimicrobial composition comprising coniferous resin acids, alcohol, auxiliary solvent, water and optionally wetting agent, and/or pH regulator. By adding an auxiliary solvent to the composition resin/rosin acids are maintained in the aqueous solution, when alcohol evaporates. The auxiliary solvent has good solubility both in water and alcohol, and it dissolves resin/rosin acids. Typically, the most suitable auxiliary solvents are those, which have evaporative properties close to water. Low toxicity profile is also one important selection criteria for an auxiliary solvent.

An advantage of the composition, method and use of the disclosure is that an improved antimicrobial composition can be obtained which creates a thin protecting film on to the treated surface. Thus, a long-lasting antimicrobial effect can be obtained. Also, another advantage of the antimicrobial composition of the disclosure is that said antimicrobial composition can be used as a base (concentrate) composition for the manufacturing of various antimicrobial products, such as tailor-made disinfectants for inanimate and living surfaces, as well as deodorizers, sanitizers and wound sprays. Said antimicrobial composition can also be used in detergents, softeners and preservatives. Still, another advantage of the antimicrobial composition of the disclosure is that by the method of the disclosure it is possible to manufacture an antimicrobial composition that is not harmful to the environment or animals including humans.

Typically, said antimicrobial composition of the disclosure can be used as a base (concentrate) composition in the manufacturing of various products including deodorizer sprays against foot sweat to be applied on shoes or foots, deodorants, hand disinfectants, wound sprays, sanitizers for example for the disinfecting of animal cages and bedding. The antimicrobial composition can also be used as a concentrate in the manufacturing of sanitizers, detergents and cleaning agents. Said composition can also be used in various industry applications including but not limited to food industry, sports equipment industry, such as skates, sports clothing and shoes, construction industry, cosmetics industry, gas and oil industry, mining industry, paper industry, chemical industry, and products as well as for consumer and professional use. One advantage of the composition is that it can be applied on any alcohol-tolerant surface. By applying said composition on non-woven surfaces it is possible to produce for example air filters having an evenly distributed antimicrobial film. Said antimicrobial composition can also be used as a base (concentrate) composition in the manufacturing of medical products as well as products for surgical applications. Said antimicrobial composition can be applied into all products related to medical applications such as surgical tools, materials and instruments. One advantage of the antimicrobial composition is that the antimicrobial composition forms a smooth surface of resin acids on the treated surfaces, and typically resin acids do not have to be added in substantial amounts due to the film formation of said antimicrobial composition. With an auxiliary solvent, a smaller amount of coniferous resin acids is needed to cover the surface.

Another advantage of the antimicrobial composition is that it is easy to dilute and dissolve both in water and alcohol.

Method is more convenient and easy to perform, because it can be performed at room temperature. Heating is not required for dissolving the ingredients into the composition.

More specifically, the antimicrobial composition according to the disclosure is characterized by what is stated in the independent claim 1.

A method for producing the antimicrobial composition according to the invention is characterized by what is stated in the independent claim 12.

The use of the antimicrobial composition of the invention is characterized by what is stated in the independent claims 19, 20 and 21.

### BRIEF DESCRIPTION OF THE DRAWINGS

In the following the disclosure will be described in greater detail by means of preferred embodiments with reference to the accompanying drawings, in which

FIG. 1a shows an electron micrograph taken from an untreated metal sheet;

FIG. 1b shows a comparative electron micrograph taken from a metal sheet treated with an alcoholic resin acid composition without an auxiliary solvent;

FIG. 1c shows an electron micrograph taken from a metal sheet treated with an antimicrobial composition according to the invention;

FIG. 2a shows an electron micrograph taken from an untreated non-woven fibrous material (filter paper);

FIG. 2b shows a comparative electron micrograph taken from non-woven fibrous material (filter paper) treated with an alcoholic resin acid composition without an auxiliary solvent;

FIG. 2c shows an electron micrograph taken from non-woven fibrous material (filter paper) treated with an antimicrobial composition according to the invention;

FIG. 3a shows an electron micrograph taken from an untreated fibrous material (cotton fabric);

FIG. 3b shows an electron micrograph taken from fibrous material (cotton fabric) treated with an alcoholic resin acid composition without an auxiliary solvent;

FIG. 3c shows an electron micrograph taken from fibrous material (cotton fabric) treated with an antimicrobial composition according to the invention.

### DETAILED DESCRIPTION OF THE DISCLOSURE

The disclosure is based on the finding that an antimicrobial composition comprising coniferous resin acids, water and solvent, wherein the solvent is an alcohol, and thereto an auxiliary solvent selected from E and P series glycol ethers, provides an evenly distributed thin film when applied on inanimate and/or living surfaces. Said antimicrobial composition can be produced by first providing coniferous resin acids, alcohol, auxiliary solvent and water, and optionally wetting agent and pH regulator; secondly mixing alcohol and water; following admixing coniferous resin acids, an auxiliary solvent, and optionally a pH regulator and wetting agent into alcohol-water solution from second step to provide an antimicrobial composition; and optionally packaging the antimicrobial composition; and thereafter optionally diluting the obtained concentrate with water, alcohol and/or a mixture thereof to obtain a coniferous resin acid composition in concentrations ranging from 0.01 to 20 weight-% (w/v), preferably from 0.02 to 0.1 weight-% (w/v), more preferably 0.04 to 0.09 weight-% (w/v).

The disclosure relates to an antimicrobial composition comprising coniferous resin acids and solvent, wherein the solvent is an alcohol, and the composition further comprises an auxiliary solvent selected from E and P series glycol ethers, and water.

In one embodiment, the amount of alcohol in the antimicrobial composition is in the range of from about 50 to about 95 weight-%, preferably in the range of about 60 to about 90 weight-%.

The antimicrobial composition comprises alcohol, which is preferably selected from ethanol, isopropanol and n-propanol and/or mixtures thereof, preferably isopropanol.

The alcohol can be selected from ethanol, isopropanol and n-propanol and/or mixtures thereof. However, when choosing the alcohol, it should be borne in mind that different alcohols have different properties, for example n-propanol has stronger odour than ethanol and isopropanol.

In an embodiment, the amount of alcohol in the alcoholic antimicrobial composition is in the range of 50 to 95 weight-%, preferably in the range of 60 to 90 weight-%. The amount of alcohol depends on the type of alcohol used. For example, the amount of ethanol is usually larger than the amount of isopropanol. The optimum amount of isopropanol is typically more than about 40 weight-%, preferably more than about 50 weight-%, and the optimum amount of ethanol is typically in the range of from about 60 to 95 weight-%, preferably about 70 weight-%.

In an embodiment, the amount of alcohol in the antimicrobial composition is in the range of about 50 to 96 weight-%, including the range being between two of the following weight-percentages 50, 51, 52, 53, 54, 55, 56, 57, 58 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 90, 91, 92, 93, 94, 95, 96 weight-%.

In one embodiment the antimicrobial composition comprises wetting agent selected from non-ionic and/or anionic surfactants, preferably from ethoxylated alcohols, more preferably from fatty alcohol ethoxylates, wherein the degree of ethoxylation is ranging from 6 to 10 moles, preferably the wetting agent is selected from C10-016 alcohol ethoxylates AE 06 to AE 10, more preferably the wetting agent is C12-C14 alcohol ethoxylate AEO7.

The amount of wetting agent is typically in the range of from about 0.001 to 0.2 weight-% of the antimicrobial composition. In one preferable embodiment, the amount of wetting agent is about 0.025 weight-% of the antimicrobial composition.

In an embodiment, the wetting agent is selected from primary alkane sulphonate (PAS) and secondary alkane sulphonate (SAS), preferably the wetting agent is C14/16 alpha olephin sulphonate. In another embodiment, the wetting agent is amine oxides.

The amount of wetting agent depends on the type of wetting agent used. For instance, amine oxides must be added even up to about three, four or five times more than fatty alcohol ethoxylates.

In one embodiment, the pH regulator is selected from amino methyl propanol (AMP), 2-hydroxy-1-propyl ethylene amine, monoethanolamine (MEA), diethanolamine (DEA) and triethanolamine (TEA) and/or mixtures thereof, preferably the pH regulator is triethanolamine (TEA). Alternatively, or additionally pH can also be adjusted with any known pH regulators and/or compositions suitable for use as pH regulators.

Typically, coniferous resin acids are better dissolved with a pH regulator, and/or the antimicrobial composition stays homogenous due to the addition of a pH regulator.

In one preferable embodiment, the pH regulator comprises water, and the amount of water in the pH regulator composition is from about 1% to about 25%.

According to another embodiment, the pH regulator comprises water in an amount of from about 0.1 to about 90 weight-%, preferably from about 1 to about 75 weight-%.

In one preferable embodiment, the amount of pH regulator is in the range of from 0.01 weight-% to 0.9 weight-%, preferably about 0.05 weight-% of the antimicrobial composition.

In an embodiment, the antimicrobial composition comprises an auxiliary solvent, which is selected from E and P series glycol ethers, preferably from E-series glycol ethers such as ethylene glycol monomethyl ether (2-methoxyethanol), ethylene glycol monoethyl ether (2-ethoxyethanol), ethylene glycol monopropyl ether (2-propoxyethanol, ethylene glycol monoisopropyl ether (2-isopropoxyethanol), ethylene glycol monobutyl ether (2-butoxyethanol), ethylene glycol monophenyl ether (2-phenoxyethanol), ethylene glycol monobenzyl ether (2-benzyloxyethanol), diethylene glycol monomethyl ether (2-(2-methoxyethoxy)ethanol), (methyl carbitol), diethylene glycol monoethyl ether (2-(2-ethoxyethoxy)ethanol, carbitol cellosolve), diethylene glycol mono-n-butyl ether (2-(2-butoxyethoxy)ethanol, butyl carbitol), and from P-series glycol ethers such as dipropylene glycol methyl ether, dipropylene glycol methyl ether acetate, dipropylene glycol n-butyl ether, dipropylene glycol n-propyl ether, propylene glycol diacetate, propylene glycol methyl ether, propylene glycol methyl ether acetate, propylene glycol n-butyl ether, propylene glycol n-propyl ether, propylene glycol phenyl ether, tripropylene glycol methyl ether, tripropylene glycol n-butyl ether or dipropylene glycol dimethyl ether and/or mixtures thereof, preferably the auxiliary solvent is diethylene glycol monoethyl ether. Said auxiliary solvent can be selected from any known E and P series glycol ethers in addition to the previously presented E and P series glycol ethers, preferably from E series glycol ethers such as diethylene glycol ethyl ether, diethylene glycol methyl ether, diethylene glycol n-butyl ether, diethylene glycol hexyl ether, diethylene glycol n-butyl ether acetate, ethylene glycol propyl ether, ethylene glycol n-butyl ether, ethylene glycol hexyl ether, ethylene glycol n-butyl ether acetate, triethylene glycol methyl ether, triethylene glycol ethyl ether, triethylene glycol n-butyl ether, ethylene glycol phenyl ether, ethylene glycol n-butyl ether mixture, and from P-series glycol ethers such as propylene glycol methyl ether, dipropylene glycol methyl ether, tripropylene glycol methyl ether, propylene glycol methyl ether acetate, dipropylene glycol methyl ether acetate, propylene glycol n-propyl ether, dipropylene glycol n-propyl ether, propylene glycol n-butyl ether, dipropylene glycol n-butyl ether, tripropylene glycol n-butyl ether, propylene glycol phenyl ether, propylene glycol diacetate, dipropylene glycol dimethyl ether. The most suitable E and P series glycol ethers are those having volatility close to water. Also, any corresponding auxiliary solvent can be used having volatility close to water.

The amount of auxiliary solvent is preferably in the range of 0.001 to 5 weight-% of the antimicrobial composition. In one preferable embodiment, the amount of auxiliary solvent is about 0.5 weight-%.

In an embodiment, the amount of auxiliary solvent is in the range of about 0.001 to 5 weight-%, including the range being between two of the following weight-percentages 0.001, 0.002, 0.003, 0.004, 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.5, 1.0, 1.5, 2, 2.5, 3, 3.5, 4, 4.5, 5 weight-%.

In one embodiment, the antimicrobial composition further comprises fragrance. Said fragrance can be selected from any known natural, organic and synthetic fragrances and/or mixtures thereof. In an embodiment the fragrance is selected from etheric oils and other natural fragrances. In one preferable embodiment, the fragrance is menthol, and/or trans-menthone.

In another preferable embodiment, the fragrance is D-limonene. The fragrance may also be grape or fragrance with the commercial name of Cedrat grape.

In one embodiment, the antimicrobial composition further comprises humectant(s), preferably selected from glycerine, propylene glycol, pentylene glycol and polyglycol and/or mixtures thereof, preferably said humectant is glycerine. Typically, such compounds are used in products wherein preserving the natural moisture of the skin is needed, such as hand disinfectants.

In one preferable embodiment, the amount of humectant is in the range of from 0.1 to 5, preferably from 1 to 2 weight-% of the antimicrobial composition.

In one embodiment, the antimicrobial composition comprises alcohol, coniferous resin acids, auxiliary solvent, water, and optionally wetting agent, and/or pH regulator. Said composition further comprises one or more compounds selected from the group consisting of emollient(s), thickener(s), biocides such as quaternary ammonium compound(s), phenoxyethanol and isothiazolines, and/or mixtures thereof. In one embodiment, the antimicrobial composition further comprises one or more ingredients selected from the group consisting of emollient(s), thickener(s), biocide(s) and/or mixtures thereof.

In one embodiment, the antimicrobial composition comprises emollient(s) selected from isopropyl myristate, isopropyl laurate, isopropyl palmitate, isopropyl oleate and isopropyl isostearate, preferably the emollient is selected from isopropyl myristate and isopropyl oleate.

In one embodiment, the amount of emollient in the antimicrobial composition is in the range of from 0.1 to 5, preferably from 0.5 to 2 weight-% (w/v).

Still, in one embodiment, the antimicrobial composition comprises alcohol, coniferous resin acids, wetting agent, auxiliary solvent, water and pH regulator. Said antimicrobial composition can further be diluted both in alcohol and water-based products since the composition dissolves in water, alcohol and/or mixtures thereof. The antimicrobial composition can be used as such or in diluted form, wherein the dilutions are performed in such a way that the resulting diluted antimicrobial composition comprises resin acids at least about 0.01 weight-%. Thus, the antimicrobial composition can form a base composition for various products including disinfectants, sanitizers, deodorizers, detergents, softeners and cleaning agents.

According to an embodiment, the antimicrobial composition comprises a gel forming agent. Preferably said gel forming agent is selected from polyacrylates, more preferably from polyacrylate-based polymers. In an embodiment, the gel forming agent is selected from synthetic high molecular weight polymers on acrylic acid with a generic name poly(acrylic acid) (PAA or Carbomer). These may be homopolymers of acrylic acid, crosslinked with an allyl ether pentaerythritol, allyl ether of sucrose or allyl ether of propylene. Preferably said gel forming agent is neutralized with TEA in pH value of about 7.0 to about 7.5.

In one embodiment, the amount of gel forming agent is in the range of from about 0.1 to about less than 1 weight-%, preferably about 0.3 to about 0.5 weight-%.

In an embodiment, the antimicrobial composition comprises water that can be derived from other ingredients or added as such.

The amount of water in the antimicrobial composition is in the range of 1.8 to 60 weight-%.

In an embodiment the amount of water in the antimicrobial composition is in the range about 1.8 to 60 weight-%, including the range being between two of the following weight-percentages 1.8, 1.9, 2.0, 2.1, 2.2, 2.3, 2.4, 2.5, 2.6, 2.7, 2.7, 2.9, 3.0, 3.1, 3.2, 3.3, 3.4, 3.5, 3.6, 3.7, 3.8, 3.9, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 weight-%.

The term “coniferous resin acids” is meant to include coniferous resin acids derived from a natural source, like rosin such as spruce resin, for example a fraction of resin acids obtained by distilling crude tall oil derived from kraft pulping processes of coniferous trees. Particularly preferably the coniferous resin acid composition comprises the following coniferous resin acids: pimaric acid, sandaracopimaric acid, dihydroabietics acids, levopimaric acid, palustric acid, isopimaric acid, 8,12-abietic acid, abietic acid, dehydroabietic acid, neoabietic acid, dehydrodehydro abietic acid.

In an embodiment, the coniferous resin acids are provided as coniferous resin acid composition.

In an embodiment, the coniferous resin acid composition comprises coniferous resin acids in at least the following ratios: palustric acid to pimaric acid 0.9:1, palustric acid to abietic acid 1:6, palustric acid to dehydroabietic acid 1:0.8, dehydroabietic acid to abietic acid 1:8, neoabietic acid to abietic acid 1:7, neoabietic acid to palustric acid 0.9:1, pimaric acid to abietic acid 1:7.

In another embodiment, the coniferous resin acid composition comprises coniferous resin acids in at least the following ratios: palustric acid to pimaric acid 1.9:1, palustric acid to abietic acid 1:4.9, palustric acid to dehydroabietic acid 1:2.7, dehydroabietic acid to abietic acid 1:1.8, neoabietic acid to abietic acid 1:11, neoabietic acid to palustric acid 1:2.2, pimaric acid to abietic acid 1:9.4.

According to an embodiment the coniferous resin acid composition comprises pimaric acid, sandaracopimaric acid, dihydroabietics acids, levopimaric acid, palustric acid, isopimaric acid, 8,12-abietic acid, abietic acid, dehydroabietic acid, neoabietic acid, dehydrodehydro abietic acid and minor amounts of other resin acids.

In an embodiment, the coniferous resin acid composition comprises the following rosin/resin acid composition: 40-50 w-% of abietic acid, 0.5-1 w-% of 8,12-abietic acid, 6-7 w-% of pimaric acid, 1-2 w-% of sandaracopimaric acid, 1-1.5 w-% of dihydroabietics acid (group), 0-0.5 w-% of levopimaric acid, 6.5-7.5 w-% of palustric acid, 6-7 w-% of neoabietic acid, 5-6 w-% of dehydroabietic acid, 0.5-1.5 w-% of isopimaric acid, and minor amounts of other resin acids. In an embodiment, the amount of palustric acid is at least 6 w-% of the rosin/resin acid composition, preferably from 6 to 10 w-%, more preferably from 7 to 8 w-%.

In another embodiment, the coniferous resin acid composition comprises the following rosin/resin acid composition: 30-40 w-% of abietic acid, 1-2 w-% of 8,12-abietic acid, 2-5 w-% of pimaric acid, 2-3 w-% of sandaracopimaric acid, 1.2-1.5 w-% of dihydroabietics acid (group), 0-0.1 w-% of levopimaric acid, 6.7-7.5 w-% of palustric acid, 3-4 w-% of neoabietic acid, 18-20.5 w-% of dehydroabietic acid, 2-4 w-% of isopimaric acid, and minor amounts of other resin acids. In an embodiment, the coniferous resin acid composition comprises 5-7 w-% of unknown rosin acids. In an embodiment, the amount of palustric acid is at least 6.5 w-% of the resin/rosin acid composition, preferably from 7 to 10 w-%, more preferably from 7 to 9 w-%.

In an embodiment, the coniferous resin acid composition comprises from 1 to 5%, preferably from 2 to 4% of unsaponifiable matter.

The acid value of the coniferous resin acid composition is typically from 160 to 180 mg KOH/g, typically about 170 mg KOH/g. The melting point of the coniferous resin acid composition is typically from 62° C. to 95° C. The fire/flash point of the coniferous resin acid composition is typically from 180° C. to 225° C. The amount of the coniferous resin acids in the coniferous resin acid composition is typically from 70 to 90 wt-%, preferably from 70 to 80 wt-%. The coniferous resin acid composition comprises typically >90 wt-%, preferably >95 w-% of free resin/rosin acids.

The coniferous resin acids are typically added as coniferous resin acid composition.

In one embodiment, the coniferous resin/rosin acids are added to the composition in an amount of about 0.01 to about 30 weight-%, preferably about 0.04 to about 10 weight-%, more preferably about 0.07 to about 2.5 weight-%. Said antimicrobial composition is typically further diluted with water or alcohol and/or mixtures thereof before or during manufacturing of end products. Such end products are typically selected from disinfectants, sanitizers, detergents, softeners, preservatives, wound sprays, cosmetics, and/or spray-on dressings.

In an embodiment, the antimicrobial composition is diluted with water, alcohol, and/or mixtures thereof. In another embodiment, the antimicrobial composition is diluted with acetone, alcohol, water, and/or mixtures thereof. Typically, the antimicrobial composition can be diluted with any known solvent suitable for dissolving said antimicrobial composition.

In an embodiment, the amount of coniferous resin acids in the diluted antimicrobial composition can range from about 0.001 to about 0.1 weight-%.

In an embodiment, the amount of coniferous resin acids is in the range of 0.01 to 30 weight-% (w/v), preferably in the range of 0.04 to 5 weight-% (w/v), more preferably in the range of 0.07 to 1.5 weight-% (w/v) of the composition.

In another embodiment, the amount of coniferous resin acids in the antimicrobial composition is in the range of about 0.01 to 30 weight-%, including the range being between two of the following weight-percentages 0.01, 0.02, 0.03, 0.04, 0.05, 0.06, 0.07, 0.08, 0.09, 0.1, 0.15, 0.2, 0.25, 0.5, 1.0, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30 weight-%.

According to an embodiment, the antimicrobial composition comprises hydroxypropyl cellulose in addition to coniferous resin acids, alcohol, auxiliary solvent, water, and optionally a wetting agent, and a pH regulator. Preferably the amount of hydroxypropyl cellulose is in the range from about 1 to about 2 weight-%, more preferably from about 1 to about 1.5 weight-%. Said antimicrobial composition can be used as spray-on dressing.

The disclosure also relates to a method for producing antimicrobial composition comprising the following steps:
a) providing coniferous resin acids, alcohol, auxiliary solvent and water, and optionally wetting agent and/or pH regulator;
b) mixing alcohol and water;
c) adding coniferous resin acids, auxiliary solvent, as well as optionally wetting agent, water and/or pH regulator into the alcohol-water solution from step b) and mixing until a clear, homogenous solution is obtained to provide an antimicrobial composition;
d) optionally packaging the antimicrobial composition; and/or
e) optionally diluting the obtained concentrate with water, alcohol and/or mixtures thereof to obtain an alcoholic coniferous resin acid composition with coniferous resin acid concentrations of more than 0.01 weight-% (w/v).

In an embodiment, step b) mixing alcohol and water is performed for about 15 minutes. Alternatively, the antimicrobial composition is manufactured by the following steps:
a) providing coniferous resin acids, alcohol, auxiliary solvent and water, and optionally wetting agent and/or pH regulator;
b) mixing alcohol, water, coniferous resin acids, auxiliary solvent in any order and optionally adding wetting agent and/or pH regulator to provide an antimicrobial composition;
c) optionally packaging the antimicrobial composition; and/or
   optionally diluting the obtained concentrate with water, alcohol and/or mixtures thereof to obtain an antimicrobial composition with coniferous resin acid concentrations of more than 0.01 weight-% (w/v). Typically, the mixing steps b) and c) of the two methods are performed for about 15 to 90 minutes, preferably for about 30 to 60 minutes. Typically, the alcohol and water solution, resin acid composition, auxiliary solvent, and optional wetting agent and/or pH regulator are mixed until a clear, homogenous solution is obtained.

Furthermore, typically since no precipitate is formed, no filtering and/or heating above 30 degrees is needed in the manufacturing of the antimicrobial composition.

According to one embodiment, the coniferous resin acids are first dissolved in alcohol, and thereafter other ingredients, such as water, auxiliary solvent, optional pH regulator and optional wetting agent, are added. However, this is not a preferable manufacturing method, as said method may result in at least a momentary precipitation of the coniferous resin acids.

The disclosure also relates to the use of the antimicrobial composition as a disinfectant. According to one preferable embodiment, the antimicrobial composition is used as disinfectant for inanimate surfaces.

The term “inanimate surface” means here all inanimate surfaces, such as surfaces of countertops, floors, walls, roofs, any objects, items and articles. The term “inanimate surface” means here all inanimate surfaces, such as hard surfaces and soft surfaces. Hard surface means all hard surfaces that can be made of any hard materials such as stone, concrete, metal, glass, plastic, rubber, hooves, nails, wood and wood-based materials, such as cardboard, plywood, wood-based products for use in construction industry, and those used in furniture, packaging and medical products, and/or mixtures thereof. Soft surface means all soft inanimate surfaces that can be made of any soft material such as fabric, fur, hair, leather, paper, plastic, textile (comprising woven and non-woven), rubber, materials of vegetable or fruit origin, and food.

In one embodiment, the antimicrobial composition is disinfectant for use on hard and/or soft inanimate surfaces, suitable for use as deodorizer, disinfectant for animal cages and bedding, disinfectant in hospitals, industrial plants and households, disinfectant for medical instruments and surgical tools and materials.

In another embodiment, the antimicrobial composition is used as detergent and/or cleaning agent for inanimate and/or living surfaces.

In another preferable embodiment, the antimicrobial composition is used as disinfectant for living surfaces.

The term “living surfaces” means here all living surfaces, for example of animal or human origin. In an embodiment disinfectant for living surfaces means all living surfaces such as surfaces of body and/or organs or parts of organs of animals and humans.

In one preferable embodiment, the living surface is skin. According to another embodiment, the antimicrobial composition is a hand disinfectant.

In another preferable embodiment, the living surface is hooves, nails and/or fur.

In one embodiment, the antimicrobial composition is for use as hand and/or body disinfectant.

In an embodiment, the antimicrobial composition is disinfectant for living surfaces, such as hand and/or body disinfectant, deodorant spray, disinfectant for wounds such as wound spray and/or disinfectant for surgical purposes.

Said antimicrobial composition is suitable for use in medical applications including but not limited to polymers such as medical tubing and catheters, dressings, bandages, clothes, plasters, tissues, towels, medical textiles, medical furniture such as counters, tables and handles, medical valves, dental medical applications such as dental bridges, implanted ports, bone cement, polymer implants, salves, ointments, lotions, wound sprays, cremes, prosthesis implants, drops, gels, skin antiseptics, sutures, suture anchors, cloves, clamps, hooks, drains, hoses, cannulas, tissue adhesives, medical wipes, medical fillers, bone substitutes, ear implants, hospital mattresses and/or pressure cuffs.

According to one embodiment, the antimicrobial composition is suitable for use as disinfectant for surfaces, preferably as disinfectant for inanimate surfaces, such as a detergent and/or cleaning agent, a deodorizer, disinfectant for animal cages and bedding, disinfectant in hospitals, industrial plants and households, disinfectant for medical instruments and/or surgical tools and materials.

Typically, the antimicrobial composition is for use in industry applications including but not limited to applications of paint industry, food industry, paper industry such as process waters, building industry such as coatings and/or mould protection, gas and oil industry. Said antimicrobial composition is also suitable for use in cosmetics, preservatives, filters, towels, cleaning wipes, mops, textiles, toothpastes, mouth washes, soaps, shampoos, washing agents, toys, plastic cutlery, sauna and/or bathroom sprays, cooking utensils, brushes, pouches, ropes, vapes (vaporizers), pipes, valves, switches, plugs, keyboards, hangers, seals, condoms, water tanks, reservoirs and/or pools. The antimicrobial composition is also suitable for use in hygiene applications, such as deodorizers, cosmetics, toothpastes, mouth washes, soaps, shampoos, washing agents, sauna and/or bathroom sprays, cleaning wipes, brushes and/or condoms.

In an embodiment the antimicrobial composition is suitable for use in hygiene applications, cleaning applications and/or process waters.

The antimicrobial composition is suitable for use in cleaning applications, such as preservatives, filters, towels, cleaning wipes, mops, textiles, detergents, softeners and/or washing agents.

According to an embodiment of the disclosure the antimicrobial composition is suitable for use in paint industry applications, for example as can and/or film preservative.

The following examples are given for further illustration of the invention without limiting the invention thereto.

### EXAMPLES

Example 1 presents a composition of a coniferous resin acid composition as well as a composition of an antimicrobial composition, and a composition of a ready to use disinfectant, and a manufacture of antimicrobial compositions. Example 2 is a comparative example presenting results from comparison of electron microscope pictures taken from metal sheets, fibrous non-woven material (filter paper) and fibrous material (fabric) treated with the antimicrobial composition according to the invention as described in Example 1, and comparative electron micrographs taken from the same materials treated with an alcoholic composition comprising resin acids but without an auxiliary solvent, and comparative electron micrographs taken from untreated sheets. Example 3 presents measurement of antibacterial activity of surfaces coated with the antimicrobial composition of Example 1. Example 3.1 presents measurement of antibacterial activity of surfaces coated with the antimicrobial composition of Example 1. Example 4 is a comparative example presenting antimicrobial activity values of surfaces coated with an alcoholic resin acid composition without wetting agent, auxiliary solvent and pH regulator. Examples 5 to 9 present products manufactured from the antimicrobial composition according to the present specification.

### Example 1

The Composition of the Coniferous Resin Acid Composition

A rosin acid composition was analysed by gas chromatography according to standard method ASTM D5974. As shown in table 1 the rosin acid composition of the coniferous resin acid composition consists mainly of abietic acid, but there are also significant amounts of pimaric acid, palustric acid, dehydroabietic acid and neoabietic acid. For example, following ratios can be calculated from table 1 values: the ratio of pimaric acid to palustric acid is 1:1.1, palustric acid to abietic acid is 1:6.4, dehydroabietic acid to abietic acid is 1:8.4, neoabietic acid to abietic acid is 1:7, neoabietic acid to palustric acid is 1:1.1, pimaric acid to abietic acid is 1:7.

TABLE 1
Rosin acid composition of the coniferous resin acid composition

| Rosin acid composition | Weight-% |
|---|---|
| Secodehydroabietic 1 | |
| 8,15-Isopimaradien-18-oic acid | 0.2 |
| Secodehydroabietic acid 2 | |
| 8,15-pimaric acid | 0.1 |
| Pimaric acid | 6.3 |
| Sandaracopimaric acid | 1.4 |
| Dihydroabietics acid (group) | 1.2 |
| Levopimaric acid | 0.2 |
| Palustric acid | 7.0 |
| 7,9 (11) -abietic acid | 0.0 |
| Isopimaric acid | 1.0 |
| 13-B-7,9 (11)-abietic acid | 0.1 |
| 8,12-abietic acid | 0.7 |
| Abietic acid | 44.7 |
| Dehydroabietic acid | 5.3 |
| Neoabietic acid | 6.4 |
| Dehydrodehydroabietic acid | 0.3 |
| Nordehydroabietic acid | |
| Unknown rosin acids | 4.0 |
| Rosin acids, total | 78.7 |
| Non-eluting compounds | 20.9 |

In addition to rosin acids shown in table 1, the coniferous resin acid composition comprised also about 20.9 w-% of non-eluting compounds.

The unsaponifiable matter of the coniferous resin acid composition was analysed according to standard method ASTM D1965. The coniferous resin acid composition comprised about 3.4% of unsaponifiable matter.

The coniferous resin acid composition was further analysed for its fatty acid composition. The coniferous resin acid composition consisted only minor amount of fatty acids, namely 0.1 weight-% of anteiso-heptadecanoic acid and 0.2 weight-% of unknown fatty acids.

The Composition of the Antimicrobial Composition

The antimicrobial composition was manufactured in a 100 litres mixer. The following ingredients were used:

Isopropyl alcohol CAS number 67-63-0

Water

Resin acid fraction (composition of the coniferous resin acid composition presented above)

Diethylene glycol monoethyl ether, CAS number 111-90-0, under trade name: Dowanol DE)

Triethanolamine, CAS number 102-71-6, under trade name: DOW Triethanolamine

Alcohol Ethoxylate C12-C14, EO7, CAS number 68439-50-9, under trade name: Rokanol L7

Manufacturing of the Antimicrobial Composition (Concentrate)

1070 kg of isopropanol was dissolved in 430 kg of water and mixed for 15 minutes until properly dissolved. Then 40 kg of resin/rosin acid composition (resin acid fraction), 200 kg of diethylene glycol monoethyl ether (Dowanol), 10 kg of fatty alcohol ethoxylate C12-C14, EO7, and 20 kg of TEA (pH regulator) was mixed into the solution and stirred until properly dissolved and a clear solution was obtained.

The resulting antimicrobial composition was packaged and stored in an ambient temperature for further use.

Disinfectant for Surfaces

Disinfectant for surfaces was prepared by diluting the antimicrobial concentrate. 150 kg of said antimicrobial concentrate was mixed with alcohol-water solution comprising 1120 kg of water and 1400 kg of isopropyl alcohol. The resulting mixture was mixed until clear and properly dissolved.

Manufacturing of the Antimicrobial Composition (Ready to Use Disinfectant)

The antimicrobial composition was manufactured in a 100 litres mixer.

49.5 kg of isopropyl alcohol and 38.5 kg of water were mixed for about 15 minutes. Then 0.1 kg of resin/rosin acid composition, 0.5 kg of Diethylene glycol monoethyl ether (serving as an auxiliary solvent), 0.05 kg of Triethanolamine (TEA serving as pH regulator) and 0.025 kg of alcohol ethoxylate C12-C14, EO7 (serving as wetting agent) were added to the water-alcohol mixture and mixed until the solution became clear.

The resulting antimicrobial composition was clear, and slightly yellow liquid. Said antimicrobial composition had pH value of 7.5 and density of 0.88 g/ml.

Loses by mixing and pumping were about 1% by volume.

The resulting antimicrobial composition was further diluted with water to obtain an antimicrobial composition with the following resin acid concentrations: 0.02 weight-%, 0.04 weight-% and 0.08 weight-% (w/v).

### Example 2

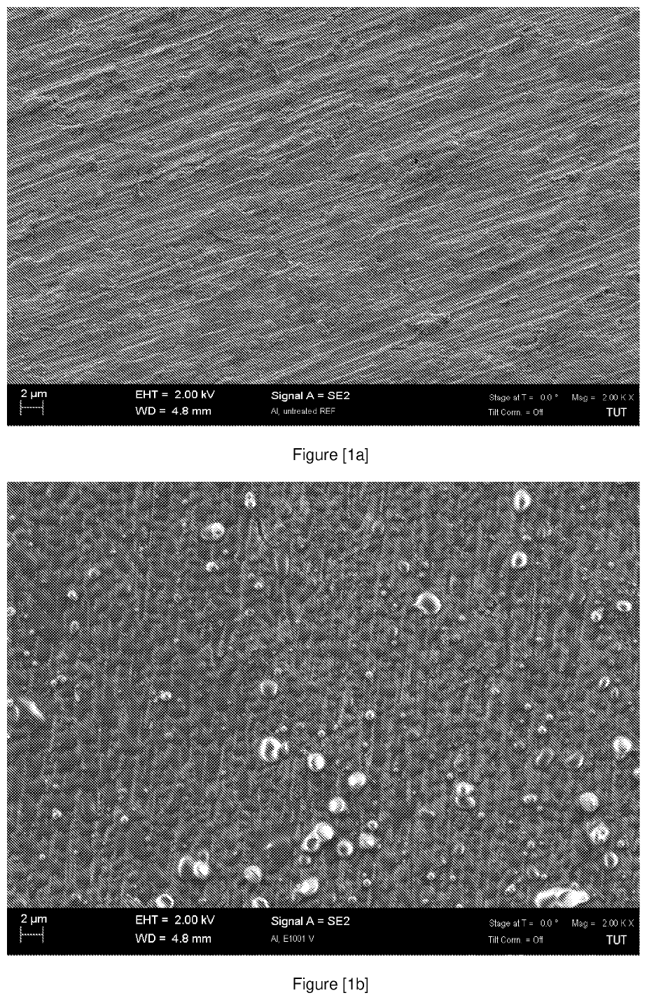

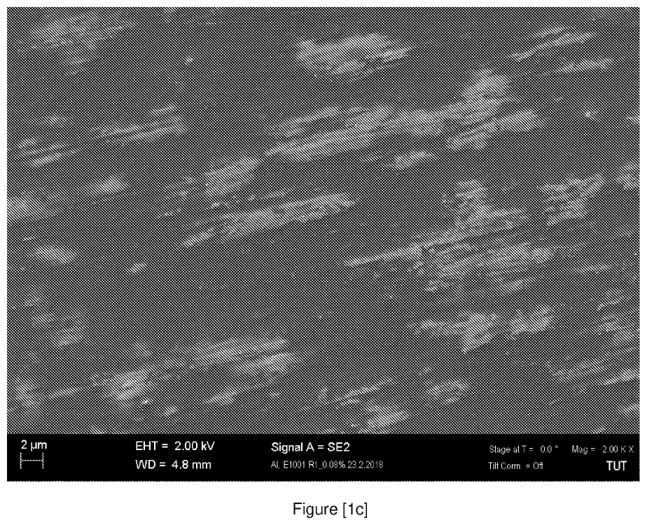

FIGS. 1a to 3c show electron micrographs taken from metal sheets treated with different coniferous resin acid compositions.

For scanning electron microscopy (SEM) studies the various substrates (metal (Al), non-woven fibrous material (filter paper), fabrics) were sprayed five consecutive times with the solutions and dried in a fume cupboard. The fabrics and wood samples were then attached to Aluminium SEM studs with carbon tape or carbon glue and coated with carbon. The SEM characterizations were conducted using Zeiss ULTRAplus, which was equipped with an ultra-high-resolution field-emission gun. The images were taken with secondary-electron (SE) detectors (SE2: traditional SE-detector outside the electron column, or InLens: located inside the electron column) using low accelerating voltage (2 kV).

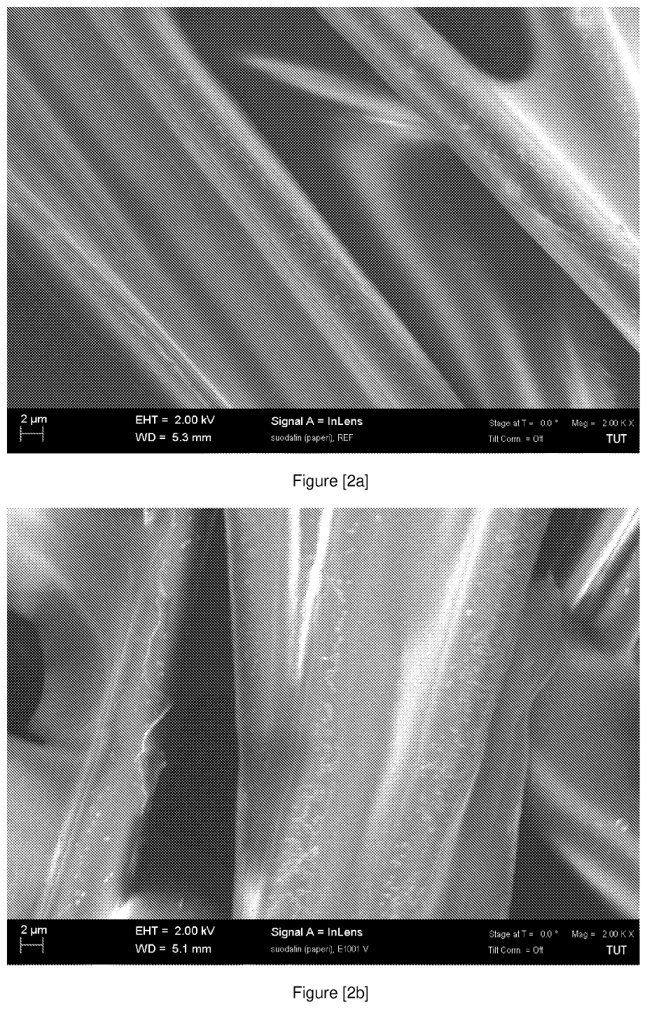

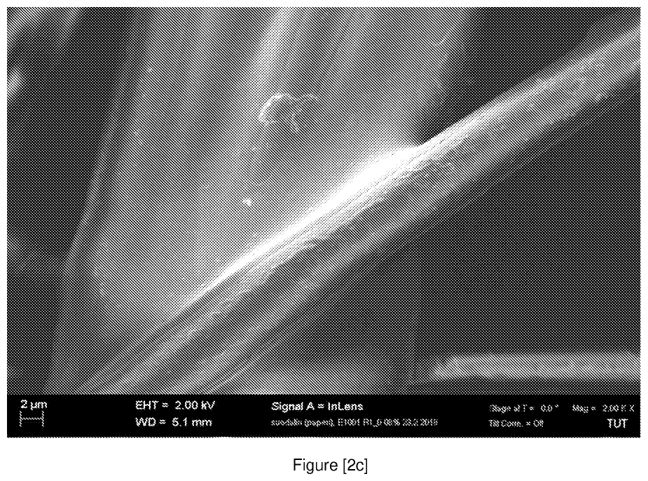

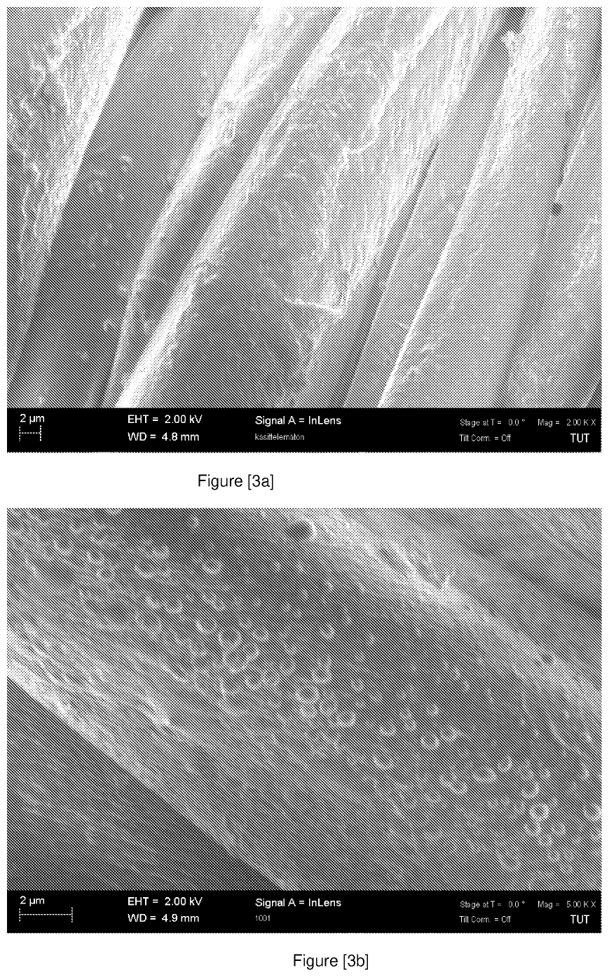

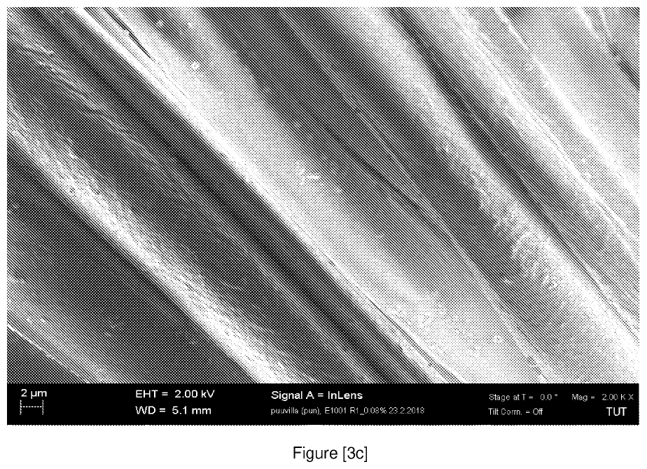

FIGS. 1a, 1b and 1c are electron micrographs taken from metal (aluminium) sheets. FIG. 1a shows an electron micrograph taken from an untreated metal (Al) sheet. FIG. 1b shows a comparative electron micrograph taken from metal sheet treated with an alcoholic resin acid composition (composition presented in Example 4) without an auxiliary solvent. FIG. 1c shows an electron micrograph taken from metal sheet treated with antimicrobial composition according to the specification. FIGS. 2a, 2b and 2c are electron micrographs taken from non-woven fibrous material (filter paper). FIG. 2a shows an electron micrograph taken from an untreated filter paper. FIG. 2b shows an electron micrograph taken from a filter paper treated with an alcoholic antimicrobial composition (of Example 4) without an auxiliary solvent. FIG. 2c shows an electron micrograph taken from a filter paper treated with an antimicrobial composition according to the specification. FIGS. 3a, 3b and 3c are electron micrographs taken from fibrous material (cotton). FIG. 3 shows an electron micrograph taken from untreated cotton. FIG. 3b shows an electron micrograph taken from cotton treated with an alcoholic antimicrobial composition (of Example 4) without an auxiliary solvent. FIG. 3c shows an electron micrograph taken from cotton treated with an antimicrobial composition according to the specification.

As can be seen from FIGS. 1c, 2c and 3c the surfaces treated with an antimicrobial composition according to the invention show a smooth and evenly distributed surface of coniferous resin acid composition, i.e. a thin film of coniferous resin acids, whereas in FIGS. 1b, 2b and 3b it can be seen that the comparative compositions do not provide an evenly distributed and smooth surfaces but instead many droplets and an uneven distribution of said coniferous resin acid compositions.

Thus, this example 2 clearly shows that it is not possible to produce a smooth thin film of coniferous resin acids on the surfaces with an alcoholic resin acid composition that does not comprise an auxiliary solvent.

### Example 3

Measurement of antibacterial activity of surfaces coated with an antimicrobial composition was studied. Test results obtained by dilutions of antimicrobial composition according to Example 1, comprising resin acids at concentrations of 0.02 weight-%, 0.04 weight-% and 0.08 weight-% (w/v) are presented. The tests were performed in accordance with method EN22196, and the tested strain was Staphylococcus aureus. Further the diluted antimicrobial composition according to Example 1 comprising coniferous resin acids at concentration 0.08 weight-% (w/v) was used and the antimicrobial test was performed in accordance with standard ISO 22196:2007.

Test Method Parameters

| | |
|---|---|
| Test | Polystyrene, the size of 1000 mm² with antimicrobial |
| surface | composition in an amount of 0.3 ml/10 cm². The surface |
| | was then dried and used as test surface. Three parallel test |
| | surfaces were prepared. |
| Control | Polystyrene with 0% of the antimicrobial composition. |
| surface | |
| Volume of | 0.1 ml (4.4 lg of bacteria) |
| inoculum | |
| Contact | 23.5 ± 0.5 h |
| time h | |
| Test | 35° C. |
| temperature | |
| Culture | Tryptone soy agar |
| media | |
| Neutralizer | Polysorbate 80 30 g/l, Sodium dodecyl sulphate 4 g/l, |
| | Lecithin 3 g/l, Sterilized in autoclave |
| Bacterial | Staphylococcus aureus ATCC 6538 |
| strain | |

0.1 ml of bacterial suspension (4.4 lg of cells/ml) was inoculated on test surfaces and covered with cover glasses. As a result, the bacterial suspension was evenly distributed over the test surface. Three parallel control samples were prepared as described above.

Thus, in total, three parallel samples were obtained for each type of surface (as a result, 3 test kits and three control sets were obtained). Then, all sets were covered with caps to protect them from drying out and were incubated in a thermostat at 35° C. for 23.5±0.5 hours.

After the incubation contact (24 hours), 10 ml of neutralizer was added to each sample.

The contents of the containers were thoroughly mixed and shaken.

The samples were held for 5 minutes.

For counting it was prepared seven of decimal dilutions of the test and control suspension. Sample of 1 ml of each dilution was taken and inoculated using the spread plate technique. Petri dishes were incubated 48 h at 36° C.

As a result, the surviving bacterial colonies were counted in control and control samples according to the formula:

N=(100·C·D·V)/A;

N—the number of viable bacteria recovered per mm² per test specimen;

C—the average plate count for the duplicate plates;

D—the dilution factor for the plates counted;

V—the volume, in ml, of neutralizer added to the specimen;

A—the surface area, in mm².

Validation Results

Validation results are presented in table 1.

N_{VO} is the amount of cfu/ml in the validation suspension of test microorganisms, only divided by 10. This is because after adding this suspension to the validation mix, the amount of cfu/ml per given mixture is reduced by a factor of 10.

TABLE 1
Validation results

| | Validation suspension | Validation control |
|---|---|---|
| Test organism | Nvo | C |
| Staphylococcus aureus | 67 | 49 |
| ATCC 6538 | | |
| | 30 ≤ Nvo ≤ 160 | C ≥ 0.5 × Nvo |

Abbreviations and Mathematical Formulas Used in the Result Table

N=(100×C×D×V)/A

where

N is the number of viable bacteria recovered per mm² of test specimen;

C is the average plate count for the duplicate plates;

D is the dilution factor for the plates counted;

V is the volume, in ml, of neutralizer added to the specimen;

A is the surface area, in mm², of the cover film.

R=(Ut−Uo)−(At−Uo)=Ut−At

where

R is the antibacterial activity;

Uo is the average of the common logarithm of the number of viable bacteria, in cells/ml, recovered from the control specimens immediately after inoculation;

Ut is the average of the common logarithm of the number of viable bacteria, in cells/ml, recovered from the control specimens after 24 h;

At is the average of the common logarithm of the number of viable bacteria, in cells/ml, recovered from the test specimens after 24 h.

Antibacterial Activity of Surfaces Coated with Antimicrobial Composition

Table 3 presents results obtained from antimicrobial compositions presented in example 1 and comprising resin acids at concentrations of 0.02 weight-%, 0.04 weight-% and 0.08 weight-% (w/v). The antimicrobial test was performed in accordance with method EN22196, and in this test the tested strain was Staphylococcus aureus. In this test the volumes of inoculum and antimicrobial composition were 0.5 ml and said sample and control plates were incubated for 24 h. The amount of antimicrobial composition in test samples was 0.07 ml/cm².

TABLE 3
Antibacterial activity of surfaces coated with antimicrobial
composition comprising resin acids at concentrations of
0.02 weight-%, 0.04 weight-% and 0.08 weight-% (w/v)

| | Resin acid concentrations of the | | | | |
|---|---|---|---|---|---|
| | antimicrobial composition | | | | |
| Dilu- | | 400 | | | |
| tion | 200 | ppm | 800 | Control 1 | |
| range | ppm | cfu/ml | ppm | cfu/ml | N in 0.5 ml |
| 1 | >300 | 137 | 13 | | |
| −1 | >100 | 37 | 0 | | |
| −2 | 15 | 0 | 0 | | |
| −3 | 0 | 0 | 0 | >300 | >200 |
| −4 | | | | >300 | 30/20 |
| −5 | | | | >100 | 2/4 |
| −6 | | | | 10/10 | 0/0 |

| | | |
|---|---|---|
| N = (100 × C × D × V)/A | N = (100 × C × | 25 × 10⁵ |
| N200 = 100 × 1500 × 1/7 | D × V)/A | 2.5 × 10⁵ |
| 2.1 × 10⁴ | 100 × 10⁶ × 1/7 = | Lg 5.4 |
| At lg 4.32 | 1.4 × 10⁷ | |
| R 200 = 7.15 − 4.32 = 2.83 | UT lg 7.15 | |
| N400 = 100 × 370 × 1/7 | | |
| 5.2 × 10³ | | |
| At lg 3.71 | | |
| R 400 = 7.15 − 3.71 = 3.44 | | |
| N800 = 100 × 13 × 1/7 | | |
| 1.86 × 10² | | |
| At lg 2.27 | | |
| R 800 = 7.15 − 2.27 = 4.88 | | |

Antimicrobial Activity Results of Test and Control Samples

Results of test and control samples are presented in table 2.

TABLE 2
Antimicrobial activity results of test and control samples

| Concentration of | | | | | | | | | |
|---|---|---|---|---|---|---|---|---|---|
| antimicrobial | | | | | | | Ut | At | R |
| composition | Dilution | Number of parallel tests | | | Average | Uo | cfu/mm² | cfu/mm² | cfu/mm² |
| in surfaces | range | 1 | 2 | 3 | cfu/ml | Lg | in lg | in lg | in lg |
| 0% | −2 | ]>300 | >300 | >300 | >300 | 4.4 | 5.3 | | |
| | −3 | >100 | >100 | 88 | >96 | | | | |
| | −4 | 20/33 | 25/31 | 4/7 | 20 | | | | |
| | −5 | 0 | 0 | 0 | 0 | | | | |
| | −6 | 0 | 0 | 0 | 0 | | | | |
| | −7 | 0 | 0 | 0 | 0 | | | | |
| 800 ppm | 1 | 13 | 11 | 15 | 13 | | | 1.3 × 10 | 4.19 |
| 0.08% | −1 | 0 | 0 | 0 | <1 | | | 1.11 | |
| | −2 | 0 | 0 | 0 | <1 | | | | |
| | −3 | 0 | 0 | 0 | <1 | | | | |
| | −4 | 0 | 0 | 0 | <1 | | | | |
| | −5 | 0 | 0 | 0 | <1 | | | | |
| | −6 | 0 | 0 | 0 | <1 | | | | |
| | −7 | 0 | 0 | 0 | <1 | | | | |

As can be seen from table 1, the test surfaces covered with the antimicrobial composition (0.08 weight-% of coniferous resin acids) possesses strong bactericidal activity, wherein the reduction was more than 4.19 lg cells/mm² for referenced strain Staphylococcus aureus ATCC 6538.

### Example 3.1

Following is presented measurement of antibacterial activity of surfaces coated with antimicrobial composition (ready to use disinfectant from Example 1). The test was performed in accordance with method EVS-EN 13697:2015.

Test Conditions

| | |
|---|---|
| Test surface | Non-porous surface (polystyrene) treated with an |
| | antimicrobial composition (ready to use disinfectant) |
| Control surface | non-porous surface |
| Exposure time s | 15 s, 30 s |
| Test temperature | 19.5° C. ± 0.5° C. |
| Interfering | clean conditions (0.3 g/l bovine albumin) |
| substance | |
| Neutralizer | Polysorbate 80 30 g/l, Sodium dodecyl sulphate 4 g/l, |
| | Lecithin 3 g/l |
| Test organisms | Staphylococcus aureus ATCC 6538, Escherichia coli |
| | ATCC 10536, Enterococcus hirae ATCC 10541, |
| | Pseudomonas aeruginosa ATCC 15442 |
| Incubation | 36.5° C. ± 0.5° C. |
| temperature | |
| Test method and | Dilution neutralization |
| its validation | |

Test Results

The validation test and antibacterial test results obtained for the antimicrobial composition (ready to use disinfectant) are presented in the following tables 4 to 11.

Escherichia coli

TABLE 4
Validation test

| | | NC | NT |
|---|---|---|---|
| | | Neutralization | Neutralization |
| Test organism | Dilution range | control | test |
| Escherichia coli | −4 | 111/110 | 98/115 |
| ATCC 10536 | −5 | 9/11 | 6.13 |
| | | 1.06 × 10⁷ | 1.07 × 10⁷ |
| | lg | 7.03 | 7.03 |

TABLE 5
Test results

| | | | | Test results | | |
|---|---|---|---|---|---|---|
| | Bacterial test | Water control | | | Concentration 100% | |
| | suspension | Dilution | | Dilution | Contact-time | |
| Test organism | N | range | Nc | range | 15 s. | 30 s. |
| Escherichia coli | −6 >200/>200 | −4 | 134/149 | 1 | 0; 0 | 0; 0 |
| ATCC 10536 | −7 30/21 | −5 | 14/17 | −1 | 0; 0 | 0; 0 |
| | 6.4 × 10⁶ | | 1.5 × 10⁷ | −2 | 0; 0 | 0; 0 |
| | | lg | 7.17 | Nd | <0.1 | <0.1 |
| | | | | Nst | 0 | 0 |
| | N 6.8 | | | R | >7.07 | >7.07 |

Staphylococcus aureus

TABLE 6
Validation test

| | | NC | NT |
|---|---|---|---|
| | | Neutralization | Neutralization |
| Test organism | Dilution range | control | test |
| Staphylococcus | −4 | 104/106 | 94/107 |
| aureus | −5 | 9/5 | 8/7 |
| ATCC 6538 | | 1.05 × 10⁷ | 1.01 × 10⁷ |
| | lg | 7.02 | 7.0 |

TABLE 7
Test results

| | | | | Test results | | |
|---|---|---|---|---|---|---|
| | Bacterial test | Water control | | | Concentration 100% | |
| | suspension | Dilution | | Dilution | Contact-time | |
| Test organism | N | range | Nc | range | 15 s. | 30 s. |
| Staphylococcus | −6 >300/>300 | −4 | 154/141 | 1 | 0; 0 | 0; 0 |
| aureus | −7 42/38 | −5 | 18/10 | −1 | 0; 0 | 0; 0 |
| ATCC 6538 | 1.0 × 10⁷ | | 1.47 × 10⁷ | −2 | 0; 0 | 0; 0 |
| | | lg | 7.17 | Nd | <0.1 | <0.1 |
| | | | | Nst | 0 | 0 |
| | N 7.0 | | | R | >7.07 | >7.07 |

Pseudomonas aeruginosa

TABLE 8
Validation test

| | | NC | NT |
|---|---|---|---|
| | | Neutralization | Neutralization |
| Test organism | Dilution range | control | test |
| Pseudomonas | −4 | 100/97 | 122/112 |
| aeruginosa | −5 | 7/8 | 9/14 |
| ATCC 15442 | | 9.8 × 10⁶ | 1.17 × 10⁷ |
| | lg | 6.99 | 7.07 |

TABLE 9
Test results

| | | | | Test results | | |
|---|---|---|---|---|---|---|
| | Bacterial test | Water control | | | Concentration 100% | |
| | suspension | Dilution | | Dilution | Contact-time | |
| Test organism | N | range | Nc | range | 15 s. | 30 s. |
| Pseudomonas | −6 >300/>300 | −4 | 88/93 | 1 | 0; 0 | 0; 0 |
| aeruginosa | −7 34/47 | −5 | 9/5 | −1 | 0; 0 | 0; 0 |
| ATCC 15442 | 1.1 × 10⁷ | | 9.05 × 10⁶ | −2 | 0; 0 | 0; 0 |
| | N 7.0 | lg | 6.96 | Nd | <0.1 | <0.1 |
| | | | | Nst | 0 | 0 |
| | | | | R | >6.86 | >6.86 |

Enterococcus hirae

TABLE 10
Validation test

| | | NC | NT |
|---|---|---|---|
| | | Neutralization | Neutralization |
| Test organism | Dilution range | control | test |
| Enterococcus hirae | −4 | 97/80 | 84/75 |
| ATCC 10541 | −5 | 7/4 | 6/4 |
| | | 8.85 × 10⁶ | 7.95 × 10⁶ |
| | lg | 6.95 | 6.9 |

TABLE 11
Test results

| | | | | Test results | | |
|---|---|---|---|---|---|---|
| | Bacterial test | Water control | | | Concentration 100% | |
| | suspension | Dilution | | Dilution | Contact-time | |
| Test organism | N | range | Nc | range | 15 s. | 30 s. |
| Enterococcus | −6 >200/>200 | −4 | 90/82 | 1 | 0; 0 | 0;0 |
| hirae | −7 15/24 | −5 | 7/6 | −1 | 0; 0 | 0;0 |
| ATCC 10541 | 4.8 × 10⁷ | | 8.6 × 10⁶ | −2 | 0; 0 | 0;0 |
| | N 6.68 | lg | 6.93 | Nd | <0.1 | <0.1 |
| | | | | Nst | 0 | 0 |
| | | | | R | >6.83 | >6.83 |

It can be seen from tables 4 to 11 that the antimicrobial composition (ready to use disinfectant) possesses strong bactericidal activity on non-porous surfaces in 15 s. at 20° C. under clean conditions (0.3 g/I bovine serum albumin) for referenced strains Escherichia coli ATCC 10536, Staphylococcus aureus ATCC 6538, Pseudomonas aeruginosa ATCC 15442 and Enterococcus hirae ATCC 10541(R≥4 lg).

### Example 4

Following is a comparative example presenting antimicrobial activity values of surfaces coated with an alcoholic composition comprising coniferous resin acids without auxiliary solvent. The test was performed in accordance with ISO 22196:2007.

Alcoholic Composition Comprising Coniferous Resin Acids

The alcoholic composition comprised the following ingredients:

70-80% (weight-%, w/v) of ethanol

≤0.03% (weight-%, w/v) of Quaternary Ammonium compounds

<1% (about 0.9 weight-%, w/v) coniferous resin acids (coniferous resin acid composition presented in Example 1)

Water

Test Method Parameters

| | |
|---|---|
| Test | Stainless steel discs Ø 20 mm, covered glasses - 18 × 18 |
| surface | mm, thickness of active substance 0.00024 g/cm² |
| | Polystyrene Petri dishes, Ø 90 mm, covered glasses - 50 × |
| | 20 mm, thickness of active substance 0.00028 g/cm² |
| | with alcoholic composition in an amount of 2.6 ± 0.2 g/m² |
| Control | Stainless steel discs and Polystyrene Petri dishes with 0% of |
| surface | the alcoholic composition comprising coniferous resin |
| | acids. |
| Volume of | 0.15 ml and 0.05 ml |
| inoculums | |
| Contact | 23.5 ± 0.5 h |
| time h | |
| Test | 35° C. |
| temperature | |
| Culture | Tryptone soy agar |
| media | |
| Neutralizer | Polysorbate 80, 30 g/l, Saponin 30 g/l, Lecithin 3 g/l |
| Bacterial | Staphylococcus aureus ATCC 6538 |
| strain | |

Test 1—Stainless Steel Discs

Sterile metal surface (stainless steel disc) Ø2.2 cm (S 3.8 cm²) was placed into the Petri dish onto the surface of hard agar, and 0.1 ml of alcoholic (ethanol) composition comprising coniferous resin acids was added onto the metal surface. On one surface it was brought about 0.0009 g of coniferous resin acids with thickness of about 0.00024 g/cm². Then ethanol was evaporated from the sample surfaces by drying on air at 35° C. for about 30 minutes. Thereafter 0.05 ml of bacterial suspension containing from 2.5×10⁵ cells/ml to 10×10⁵ cells/ml was brought to the surface, and immediately after that the surface was covered with a sterile 10×18 mm (3.24 cm2) piece of glass. Then the Petri dishes were covered by a lid and incubated in thermostat at 35° C. for 23.5±0.5 hours. 4 parallel testing samples were made in total, and at the same time control samples were prepared as described above but without the addition of alcoholic composition comprising coniferous resin acids. As with test samples, 4 parallel control samples were made in total. During 24 hours of incubation, covering glasses were removed from the surface and neutralizer was added in a quantity of 1 ml on 1 stainless steel disc, washout was made with an automatic pipette on the surface of agar in the same dish. Then Petri dishes were covered by a lid and incubated in the thermostat at 35° C. for 24 hours. Survived bacterial colonies were calculated in tested and control samples by the following formula:

N=(100×C×D×V)/A,

wherein

N is the number of viable bacteria recovered per cm² per test specimen;

C is the average plate count for the duplicate plates;

D is the dilution factor for the plates counted;

V is the volume, in ml, of neutralizer added to the specimen;

A is the surface area, in mm², of the cover.

Test 2—Polystryrene Petri Dishes

2.0 ml of alcoholic (ethanol) composition was evenly brought onto the surface of the bottom of Petri dishes. On one surface it was brought about 0.0018 g of coniferous resin acids with thickness of 0.00028 g/cm². After that ethanol was evaporated from the sample surfaces by drying on air at 35° C. for about 30 minutes. Thereafter 0.15 ml of bacterial suspension containing from 2.5×10⁵ cells/ml to 10×10⁵ cells/ml was brought to the surface, and immediately after that the surface was covered with a sterile 50×20 mm (10 cm²) piece of glass. Then the Petri dishes were covered by a lid and incubated in thermostat at 35° C. for 23.5±0.5 hours. 4 parallel testing samples were made in total, and at the same time control samples were prepared as described above but without the addition of alcoholic composition comprising coniferous resin acids. As with test samples, 4 parallel control samples were made in total. During 24 hours of incubation, covering glasses were removed from the surface and neutralizer was added in a quantity of 1 ml onto 1 surface with active mixing by automatic pipette. It was kept for 5 minutes for neutralization of coniferous resin acids. Then on it was poured agar agent (Tryptone Soy agar) chilled to 45° C. Finally, the Petri dishes were covered by a lid and incubated in the thermostat at 35° C. for 24 hours. The survived bacterial colonies were calculated in tested and control samples by the same formula as described previously.

Validation of the Method

Table 4 presents the results of the method validation.

TABLE 4
Method validation

| | Validation suspension | Validation control |
|---|---|---|
| Test organism | Nvo | C |
| Staphylococcus aureus | 157 | 137 |
| ATCC 6538 | | |
| | 30 ≤ Nvo ≤ 160 | C ≥ 0.5 × Nvo |

Antibacterial Activity—Results

Results of the test and control samples are presented in table 5. The antibacterial activity R was calculated as in Example 1. The average number of viable bacteria recovered immediately after inoculation from the untreated test specimens shall be within the range 6.2×10³ cells/cm² to 2.5×10⁴ cells/cm². The number of viable bacteria recovered from each untreated test specimen after incubation for 24 h shall not be less than 6.2×10¹ cells/cm².

TABLE 5
Results of the test and control samples

| | | No of | Uo | | Ut | | At | | |
|---|---|---|---|---|---|---|---|---|---|
| Test | | tests | Cells/cm² lg | | Cells/cm² lg | | Cells/cm² lg | | R |
| Test 1 | Control | 4 | 6.7 × 10³ | 3.82 | 350 | 2.54 | | | |
| (stainless | | | | | 3.5 × 10² | | | | |
| steel discs) | Test | 4 | 6.7 × 10³ | 3.82 | | | 0 | <0.1 | >2.44 |
| Test 2 | Control | 4 | 1.5 × 10⁴ | 4.17 | 600 | | | | |
| (polystryrene | | | | | 6.0 × 10² | | | | |
| Petri dishes) | Test | 4 | 1.5 × 10⁴ | 4.17 | | | 0 | <0.1 | >2.67 |

In this example, the role of alcohol was to transfer coniferous resin acids into the surface. Said alcohol was evaporated, and this example only tested the ability of said alcoholic resin acid composition to form an antimicrobial film on the tested surfaces. However, the results clearly show that the antimicrobial film formed by the alcoholic antimicrobial composition was rather poor. As is seen from table 5, the antimicrobial activity values are rather low, and there is no significant difference between the tests performed on different surfaces i.e. stainless steel discs (Test 1) and polystyrene Petri dishes (Test 2).

### Example 5

Disinfectant for Surfaces

A disinfectant for surfaces was manufactured by first mixing 49.5 kg of isopropanol with 38.5 kg of water for about 15 minutes, following adding 0.09 kg of resin acid composition (composition was presented in example 1), and 0.5 kg of diethylene glycol monoethyl ether as an auxiliary solvent. 0.025 kg of C12-14 fatty alcohol ethoxylate and 0.05 kg of triethanolamine were added and the mixture was stirred until clear solution was obtained.

The resulting disinfectant product was suitable for use as disinfectant for surfaces, especially for alcohol tolerant surfaces.

### Example 6

Deodorizer

Deodorizer was manufactured by first mixing 58.0 kg of ethanol with 40 kg of water for about 15 minutes, following adding 0.05 kg of resin acid composition (composition was presented in example 1), and 1.0 kg of dipropylene glycol methyl ether (DPM) as an auxiliary solvent. 0.03 kg of C12-C14 fatty alcohol ethoxylate, 0.05 kg of triethanolamine, and 0.3 kg of trans-menthone were added and the mixture was stirred until a clear solution was obtained.

### Example 7

Disinfectant for Medical Applications

A disinfectant for surgical applications was manufactured by first mixing 60.0 kg of ethanol with 40.0 kg of water for about 15 minutes, following adding 0.07 kg of resin acid composition (from example 1), and 0.8 kg of diethylene glycol monoethyl ether as an auxiliary solvent. Thereafter 0.03 kg of C12-C14 alcohol ethoxylate, 0.06 kg of triethanolamine, 1.3 kg of glycerine, and 1.0 kg of isopropyl myristate were added and the solution was mixed until a clear solution was obtained.

The resulting disinfectant was suitable for use as disinfectant for medical applications, especially for surgical applications.

### Example 8

Disinfectant Gel

A disinfectant gel was manufactured by first preparing two solutions a) and b). The manufacturing process comprised the following steps:
a) 50 kg of isopropyl alcohol was mixed with 8 kg of water for about 15 minutes, following adding 0.06 kg of resin acid composition (presented in example 1), 0.5 kg of diethylene glycol monoethyl ether, 1.0 kg of isopropyl myristate, and 0.0025 kg of C12-C14 fatty alcohol ethoxylate EO7, the resulted solution was mixed until clear solution was obtained.
b) 0.3 kg of Carbomer (CAS 9003-01-4) was dispersed into 32 kg of water and mixed at moderate speed until Carbomer was hydrolysed into water.
c) Solutions a) and b) were mixed until clear solution was obtained.
d) 0.3 kg of amino methyl propanol (AMP) was slowly added to the mixture c) with vigorous stirring.

The resulting disinfectant gel had a viscosity of 15000 to 20000 cP. It was smooth, homogenous gel and suitable for use as hand disinfectant.

### Example 9

Wound Spray

A wound spray was manufactured by mixing 50.0 kg of isopropyl alcohol with 40 kg of water for about 15 minutes, following adding 0.08 kg of resin acid composition (presented in example 1), 0.5 kg of diethylene glycol monoethyl ether as an auxiliary solvent, 0.03 kg of C12-C14 fatty alcohol ethoxylate EO 07, and 0.06 kg of triethanolamine until clear solution was obtained. Thereafter, 1.2 kg of Klucel™ (hydroxypropylcellulose) was added, and the resulting mixture was mixed until smooth composition was obtained. Lastly, the product was packaged and stored at ambient temperature.

The resulting product was suitable for use as wound spray.

1. An antimicrobial composition comprising coniferous resin acids and solvent, characterized in that the solvent is an alcohol and the antimicrobial composition further comprises water and an auxiliary solvent selected from E and P series glycol ethers.

2. An antimicrobial composition comprising coniferous resin acids, characterized in that the amount of alcohol in the antimicrobial composition is in the range of from about 50 to about 95 weight %.

3. An antimicrobial composition comprising coniferous resin acids, characterized in that the amount of an auxiliary solvent is in the range of from 0.001 to 5 weight-% of the antimicrobial composition.

4. The antimicrobial composition of claim 1, characterized in that the amount of coniferous resin acids is in the range of 0.01 to 30 weight-% (w/v) of the composition.

5. The antimicrobial composition according to claim 1, characterized in that the antimicrobial composition comprises an alcohol selected from ethanol, isopropanol and n-propanol and/or mixtures thereof.

6. The antimicrobial composition according to claim 1, characterized in that said antimicrobial composition further comprises a wetting agent which is a surfactant.

7. The antimicrobial composition according to claim 6, where the surfactant is an ethoxylated alcohol having a degree of ethoxylation is ranging from 6 to 10 moles.

8. The antimicrobial composition according to claim 1, characterized in that said antimicrobial composition further comprises a pH regulator which is selected from aminomethyl propanol (AMP), 2-hydroxy-1-propyl ethylene amine, monoethanolamine (MEA), diethanolamine (DEA) and triethanolamine (TEA) and/or mixtures thereof.

9. The antimicrobial composition according to claim 1, where the E-series glycol ethers are at least one of ethylene glycol monomethyl ether (2-methoxyethanol), ethylene glycol monoethyl ether (2-ethoxyethanol), ethylene glycol monopropyl ether (2-propoxyethanol, ethylene glycol monoisopropyl ether (2-isopropoxyethanol), ethylene glycol monobutyl ether (2-butoxyethanol), ethylene glycol monophenyl ether (2-phenoxyethanol), ethylene glycol monobenzyl ether (2-benzyloxyethanol), diethylene glycol monomethyl ether (2-(2-methoxyethoxy)ethanol), (methyl carbitol), diethylene glycol monoethyl ether (2-(2-ethoxyethoxy)ethanol, carbitol cellosolve), and diethylene glycol mono-n-butyl ether (2-(2-butoxyethoxy)ethanol, butyl carbitol), and the P-series glycol ethers are at least one of dipropylene glycol methyl ether, dipropylene glycol methyl ether acetate, dipropylene glycol n-butyl ether, dipropylene glycol n-propyl ether, propylene glycol diacetate, propylene glycol methyl ether, propylene glycol methyl ether acetate, propylene glycol n-butyl ether, propylene glycol n-propyl ether, propylene glycol phenyl ether, tripropylene glycol methyl ether, tripropylene glycol n-butyl ether, dipropylene glycol dimethyl ether, and mixtures thereof.

10. The antimicrobial composition according to claim 1, characterized in that said composition further comprises fragrance.

11. The antimicrobial composition according to claim 1, characterized in that said composition further comprises a humectant.

12. The antimicrobial composition according to claim 11 where the humectant is glycerine, propylene glycol, pentylene glycol or polyglycol or mixtures thereof.

13. The antimicrobial composition according to claim 1, characterized in that said composition further comprises one or more ingredients selected from the group consisting of emollient(s), thickener(s), biocide(s) and mixtures thereof.

14. A method for manufacturing of an antimicrobial composition of claim 1, characterized in that the method comprises the following steps:
a) providing coniferous resin acids, alcohol, auxiliary solvent and water, and optionally wetting agent and/or pH regulator;
b) mixing alcohol and water;
c) adding coniferous resin acids, auxiliary solvent, as well as optionally wetting agent, water and/or pH regulator into the alcohol-water solution from step b) and mixing until a clear, homogenous solution is obtained to provide an antimicrobial composition;
d) optionally packaging the antimicrobial composition; and/or
e) optionally diluting the obtained concentrate with water, alcohol and/or mixtures thereof to obtain an alcoholic coniferous resin acid composition with coniferous resin acid concentrations of more than 0.01 weight-% (w/v).

15. The method according to claim 14, characterized in that the antimicrobial composition is diluted with water, alcohol and/or mixtures thereof.

16. The method according to claim 14, characterized in that said wetting agent is selected from ethoxylated alcohols, wherein the degree of ethoxylation is ranging from 6 to 10 moles.

17. The method according to claim 14, characterized in that said pH regulator is selected from aminomethyl propanol (AMP), 2-hydroxy-1-propyl ethylene amine, monoethanolamine (MEA), diethanolamine (DEA) and triethanolamine (TEA) and/or mixtures thereof.

18. The method according to claim 14, characterized in that said auxiliary solvent is selected from E and P series glycol ethers, where the E-series glycol ether is at least one of ethylene glycol monomethyl ether (2-methoxyethanol), ethylene glycol monoethyl ether (2-ethoxyethanol), ethylene glycol monopropyl ether (2-propoxyethanol, ethylene glycol monoisopropyl ether (2-isopropoxyethanol), ethylene glycol monobutyl ether (2-butoxyethanol), ethylene glycol monophenyl ether (2-phenoxyethanol), ethylene glycol monobenzyl ether (2-benzyloxyethanol), diethylene glycol monomethyl ether (2-(2-methoxyethoxy)ethanol, (methyl carbitol), diethylene glycol monoethyl ether (2-(2-ethoxyethoxy)ethanol, carbitol cellosolve), diethylene glycol mono-n-butyl ether (2-(2-butoxyethoxy)ethanol, butyl carbitol), and where the P-series glycol ethers is at least one of dipropylene glycol methyl ether, dipropylene glycol methyl ether acetate, dipropylene glycol n-butyl ether, dipropylene glycol n-propyl ether, propylene glycol diacetate, propylene glycol methyl ether, propylene glycol methyl ether acetate, propylene glycol n-butyl ether, propylene glycol n-propyl ether, propylene glycol phenyl ether, tripropylene glycol methyl ether, tripropylene glycol n-butyl ether or dipropylene glycol dimethyl ether or mixtures thereof.

19. (canceled)

20. (canceled)

21. (canceled)

22. The method according to claim 14, characterized in that said alcohol is selected from ethanol, isopropanol and n-propanol and/or mixtures thereof.

23. The method according to claim 14, characterized in that the amount of coniferous resin acids in the antimicrobial composition comprising coniferous resin acids is in the range of 0.01 to 30 weight-% (w/v) of the composition.
